(19) United States
(12) Patent Application Publication
(10) Pub. No.: US 2012/0093738 A1
(43) Pub. Date: Apr. 19, 2012

(54) TASTE-MASKED ORAL FORMULATIONS OF INFLUENZA ANTIVIRALS

(75) Inventors: Pratibha Sudhir Pilgaonkar, Mumbai (IN); Maharukh Tehmasp Rustomjee, Mumbai (IN); Anilkumar Surendrakumar Gandhi, Mumbai (IN)

(73) Assignee: Rubicon Research Private Limited, Mumbai (IN)

(21) Appl. No.: 13/377,740

(22) PCT Filed: Jun. 10, 2010
(86) PCT No.: PCT/IN10/00398

§ 371 Date: Dec. 12, 2011

(30) Foreign Application Priority Data
Jun. 11, 2009 (IN) 1405/MUM/2009

(51) Int. Cl. A61K 31/215; A61K 9/68; A61K 31/13; A61P 31/16; A61K 38/14; A61K 31/522; A61K 31/7072; A61K 31/708; A61K 31/43; A61K 31/7056; A61K 9/46; A61K 31/616

(52) U.S. Cl. 424/44; 514/529; 424/48; 514/659; 514/662; 514/162; 514/263.34; 514/50; 514/45; 514/198; 514/24; 514/3.7

(57) ABSTRACT

The present invention relates to taste-masked oral formulations of influenza antivirals. The taste-masked pharmaceutical formulations for oral administration comprise one or more influenza antivirals, at least one taste-masking agent and at least one pharmaceutically acceptable excipient. Further, the taste-masked influenza antiviral formulations of the present invention are provided in the form of dispersible tablets, effervescent tablets, orally disintegrating tablets, chewable tablets, bite-dispersion tablets or the like, wherein the bitter taste of influenza antivirals is masked thereby providing palatable formulations.

### FIELD OF THE INVENTION

The present invention relates to taste-masked oral formulations of influenza antivirals. Particularly, the invention relates to taste-masked formulations of influenza antivirals suitable for oral administration in the form of dispersible tablets, effervescent tablets, orally disintegrating tablets, chewable tablets, bite-dispersion tablets or the like, wherein the bitter taste of influenza antiviral is masked thereby providing palatable formulations.

### BACKGROUND OF THE INVENTION

Influenza is a viral infection that affects mainly the nose, throat, bronchi and, occasionally, the lungs. It is characterized by fever, muscle aches, sore throat, headache, nonproductive cough and rhinitis. Most infected people recover within one to two weeks. However, in the very young, the elderly, and those with other serious medical conditions, the infection can cause severe complications leading to hospitalization and death.

Influenza viruses belong to the orthomyxovirus group of RNA viruses and are divided into types designated as A, B and C. Influenza type A virus causes frequent epidemics and periodic pandemics. Type B causes local epidemics of respiratory illness. Type C infection usually causes either a very mild respiratory illness or no symptoms at all; it does not cause epidemics and hence does not have severe public health impact as with influenza types A and B. The surface glycoproteins of these influenza viruses continually undergo antigenic variations. This constant antigenic change often enables the virus to evade the immune system of the host (humans, birds, and other animals) so that the host is susceptible to changing influenza virus infections throughout life.

Influenza type A and B viruses are known to contain two surface glycoproteins called hemagglutinin (H) and neuraminidase (N). Influenza type A viruses have 16 known H subtypes and nine known N subtypes. These surface proteins can occur in many combinations that infect humans, animals and birds. Influenza C viruses are less well characterized but are known to contain a single surface hemagglutinin-esterase (HE protein) glycoprotein that exhibits hemagglutination and esterase activity. The hemagglutinin of influenza A and B viruses mediates attachment of virions to sialic acid-containing cell surface receptors of the respiratory epithelial cells. Once the virus has bound to its host cell, it is transported into the cytoplasm in an endosome. The acidic pH in the endosome activates or opens an ion channel called the M2 protein (virus-encoded matrix-2 protein), permitting hydrogen ions to enter the virion. The resulting acidification of the virus is necessary for viral uncoating, an essential step in viral replication. Post replication of the viral RNA, neuraminidase, a viral enzyme that cleaves the neuraminic acid component of sialic acid in the respiratory epithelial cell hemagglutinin receptors, acts so that the new progeny of influenza virions bud off the host cell membrane and infect other cells. Neuraminidase also prevents the formation of viral aggregates after release from host cells, and possibly facilitates viral invasion of the upper airways.

Vaccines for prevention of infection and use of antivirals either prophylactically or for treatment are currently available strategies to reduce influenza disease. Virus mutability with antigenic shifts and drifts generally makes the available influenza vaccines less effective against emergent antigenic variants of the virus. Further, influenza vaccine is also not very effective in elderly and immunosuppressed patients. Antiviral drugs may therefore be used, in such situations and during sudden influenza outbreaks, to attenuate the infection and reduce morbidity and mortality as well. These influenza antiviral drugs are used as an adjunct to vaccines in the control of influenza and generally represent a first line of defense against a new pandemic, allowing the control of the infection until sufficient quantities of a suitable vaccine can be produced.

Antivirals available for the prevention and treatment of influenza virus infection mainly include amantadine, rimantadine, oseltamivir, zanamivir, peramivir or salts or prodrugs thereof. Amantadine is effective against all influenza A virus types. It binds to the transmembrane region of the M2 protein and blocks the membrane pore, thereby preventing the uncoating process and replication. Rimantidine is also an M2 ion channel inhibitor which inhibits the replication specifically of influenza A viruses. Oseltamivir phosphate and zanamivir, currently available for the prevention of influenza virus infection, target the active site of the neuraminidase protein, thus inhibiting its sialidase activity essential for virus release. They are effective against both influenza A and B viruses.

These influenza antiviral medications are used in the prevention and treatment of influenza illness in adults, adolescents, children ≧1 year of age and if required for infants <1 year of age. They are also recommended for all hospitalized patients with confirmed, probable and suspected influenza and for treatment of suspected infections caused by the virus in individuals at higher risk for influenza complications (e.g., children <5 years of age, adults ≧65 years of age, individuals with certain chronic diseases or immunosuppressed individuals). Administering antiviral medication to these groups of patients necessitates that the medication be provided in patient-friendly dosage forms, which are easy to swallow and improve patient compliance.

Currently available influenza antiviral medications for oral administration are in the form of tablets, capsules, syrups, and dry multi-dose suspensions for reconstitution. With liquid dosage forms, such as syrups or suspensions, administration of the drug, accuracy of dosing and stability of the dosage form is a major concern. An ideal dosage form would hence be the one that provides the convenience of a tablet formulation while also providing the ease of swallowing presented by a liquid formulation; such as dispersible tablets, effervescent tablets, orally disintegrating tablets, chewable tablets, bite-dispersion tablets or the like. Such dosage forms are stable and also provide more accurate dosing than that provided by oral syrups or suspensions or their reconstituted multi-dose forms.

Further, taste is an important parameter governing the compliance of patients of any age group. The unpleasant or bitter taste or taste alteration associated with influenza antivirals can lead to low patient compliance. Therefore, in order to use them in convenient solid dosage forms that disintegrate rapidly in the mouth or disperse rapidly in an aqueous medium, taste-masking becomes very crucial.

Outbreaks of influenza occur every year and as a part of epidemic and pandemic preparedness, these influenza antivirals are stockpiled. With oseltamivir, in case were the oral suspension is out of stock; they are extemporaneously prepared from capsules or the bulk active itself, thereby indicating the importance that this antiviral has in epidemic or pandemic situations. Therefore, it becomes essential that such critically useful influenza antivirals, be presented in dosage forms that are not only pleasant tasting and easy to administer but also have good stability during shelf life. Unlike, reconstituted multi-dose suspensions, e.g., of oseltamivir phosphate that need to be used within 10 days, there exists a need for dosage forms which are stable and can be used over longer durations of time.

Several attempts have been made to develop stable, easy to administer dosage forms for these influenza antivirals. European Patent Application 2005945A2 relates to oseltamivir phosphate granules and preparation method thereof. The said granules comprise the active, diluent, binder and optionally edible flavoring essence, sweetener and/or edible pigment and are water-soluble in nature. European Patent Application 1987825A1 also discloses a pharmaceutical composition containing oseltamivir phosphate. The formulation is said to have improved preservation stability and contains one or more specific excipients selected from sugar and sugar alcohols in which equilibrium water content is 1% by weight or less at 25° C. and at 70% relative humidity and the amount of each of glucose and mannose contained in the sugars and sugar alcohols as impurities is 0.01% by weight or less. These formulations employ high amounts of more than 75% by weight of the composition of sugars or sugar alcohols.

Though dosage forms such as dry syrup, powder or granules are the especially preferred forms in the above patent applications, these dosage forms have certain drawbacks. For administration of a single therapeutic dose, a large amount of granules/powder needs to be administered, which can be inconvenient to the patient. Further, granules/powders employing more than 75% by weight of the composition of sugars or sugar alcohols are bulky to carry and can result in possible inaccuracies with the dose administered. With dry syrups, issues with respect to stability of the active can arise upon reconstitution. There exists a need, therefore, for influenza antivirals in a taste-masked form, which would obviate the necessity to administer large quantities of sugar or sugar alcohol based granules and improve the convenience and accuracy of dose administration. Further, good water solubility of these influenza antivirals, makes taste-masking highly challenging and special efforts are required to avoid solubilization of the active in the saliva to prevent perception of any bitter or unpleasant taste.

It is also required that taste-masked influenza antiviral formulations be presented in dosage forms such as orally disintegrating tablets, chewable tablets, dispersible tablets, effervescent tablets, bite-dispersion tablets or the like that increase patient compliance. There, thus, exists an urgent need for patient friendly, taste-masked influenza antiviral formulations that not only provide excellent taste characteristics, but also do not compromise on the stability, desired in vitro release profile of the active and mechanical strength of the dosage form.

After rigorous experimentation, it was surprisingly found that the taste of influenza antivirals can be masked with taste-masking agents using simple, cost-effective, easy to scale-up processes to obtain taste-masked influenza antiviral formulations which can be provided in the form of palatable dosage forms that have adequate mechanical strength, stability, desired taste and in vitro release profile.

### SUMMARY OF THE INVENTION

The present invention relates to taste-masked pharmaceutical formulations for oral administration comprising one or more influenza antiviral/s, at least one taste-masking agent and at least one pharmaceutically acceptable excipient. Further, the taste-masked influenza antiviral formulations of the present invention are provided in the form of dispersible tablets, effervescent tablets, orally disintegrating tablets, chewable tablets, bite-dispersion tablets or the like, wherein the bitter taste of the influenza antiviral is masked thereby providing palatable formulations.

### DETAILED DESCRIPTION OF THE INVENTION

Palatability and mouth feel are extremely important factors for achieving total compliance of patients who are being administered an unpleasant, disagreeable or objectionable-tasting active pharmaceutical agent. Several taste-masking technologies are known. Not all taste-masking technologies, however, work with every drug. Taste-masking technologies and processes employed to achieve the same can, in certain instances, interfere with disintegration, affect stability, provide inadequate taste-masking for a given active or interfere with the bioavailability or pharmacokinetic properties of the drug. Therefore, it becomes important to develop taste-masking technology for an active agent that not only enhances the organoleptic properties of the dosage form containing the same, but also does not interfere with the bioavailability of the drug.

The present invention discloses influenza antiviral formulations wherein the bitter, unpleasant or otherwise undesirable taste of the active is masked without compromising on the stability, handling characteristics, mechanical strength and in vitro release profile of the formulations, using simple and cost effective processes.

The taste-masked influenza antiviral formulations of the present invention comprise one or more influenza antiviral/s, at least one taste-masking agent and at least one pharmaceutically acceptable excipient.

The term “influenza antiviral/s”, as employed herein refers to any compound that can be employed for the prevention and/or treatment of any confirmed, probable and suspected influenza virus infection. “Influenza antiviral” according to the present invention includes, but is not limited to, oseltamivir, zanamivir, peramivir, amantadine, rimantadine in the form of free base or pharmaceutically acceptable salts, prodrugs, active metabolites, polymorphs, solvates, hydrates, enantiomers, optical isomers, and tautomers or racemic mixtures thereof.

Pharmaceutically effective amount of influenza antivirals are employed in the formulations of the present invention. The term “effective amount” refers to an amount effective to achieve desired preventive, therapeutic and/or beneficial effect. In one embodiment the amount of influenza antiviral in the formulation is from about 0.1 weight % to about 50 weight % based on the total weight of the formulation. In another embodiment the amount of influenza antiviral in the formulation is from about 1 weight % to about 40 weight % based on the total weight of the formulation. In a further embodiment the influenza antiviral in the formulation is administered at a dose of about 1 mg to about 500 mg, depending on the influenza antiviral employed. In yet another embodiment the influenza antiviral employed is oseltamivir in the form of free base or its pharmaceutically acceptable salt or prodrug like oseltamivir phosphate or its polymorphs, solvates, hydrates, active metabolites, enantiomers, optical isomers, tautomers or racemic mixtures. Oseltamivir phosphate is an inactive prodrug until hydrolyzed by hepatic esterases to oseltamivir carboxylate, the active metabolite and neuraminidase inhibitor.

The influenza antiviral of the present invention may be in the form of, but not limited to, powder, granules, pellets, beads, minitablets or the like. Influenza antiviral granules may be prepared by wet granulation, melt granulation, dry granulation or roll compaction or the like. In one aspect of the present invention, pellets of influenza antiviral may be prepared using extrusion spheronization. In another aspect of the present invention, influenza antiviral can be loaded on an inert carrier before taste-masking. The inert carrier can be selected from, but not be limited to, beads, pellets, spheres or similar particles that do not contain an active ingredient. Non-limiting examples of inert carriers include microcrystalline cellulose, sugar or silicon dioxide. In yet another embodiment, influenza antiviral in powder form may be treated with a taste-masking agent.

Taste-masking agents employed for the purpose of the present invention include, but are not limited to, polymeric and/or non-polymeric pharmaceutically acceptable excipients, cyclodextrins, ion exchange resins, carbomers, adsorbents, sugar substitutes, or any combinations thereof. The bitter or unpleasant taste of influenza antiviral is masked using pharmaceutically acceptable taste-masking agents by methods including, but not limited to, coating, physical mixing, melt granulation, complexation, adsorption, salt formation or the like.

Polymeric pharmaceutically acceptable excipients suitable for formulations of the present invention include, but are not limited to, cellulose derivatives, saccharides or polysaccharides, polyhydric alcohols, poly(oxyethylene)-poly(oxypropylene) block copolymers (poloxamers), vinyl derivatives or polymers or copolymers thereof, acrylic acid derivatives and the like or any combinations thereof.

Cellulose derivatives include, but are not limited to, ethyl cellulose, methylcellulose, hydroxypropylmethylcellulose (HPMC), hydroxypropyl cellulose (HPC), hydroxyethyl cellulose, hydroxymethyl cellulose, hydroxypropyl ethylcellulose, carboxymethylethyl cellulose, carboxy ethylcellulose, carboxymethyl hydroxyethylcellulose, hydroxyethylmethyl carboxymethyl cellulose, hydroxyethyl methyl cellulose, carboxymethyl cellulose, methylhydroxyethyl cellulose, methylhydroxypropyl cellulose, carboxymethyl sulfoethyl cellulose, sodium carboxymethyl cellulose, cellulose acetate, cellulose acetate phthalate, cellulose acetate butyrate, hydroxypropylmethylcellulose acetate succinate, hydroxypropylmethylcellulose phthalate, hydroxymethyl ethylcellulose phthalate, cellulose acetate phthalate, cellulose acetate succinate, cellulose acetate maleate, cellulose acetate trimelliate, cellulose benzoate phthalate, cellulose propionate phthalate, methylcellulose phthalate, ethylhydroxy ethylcellulose phthalate, or combinations thereof.

Saccharides or polysaccharides include, but are not limited to, guar gum, xanthan gum, gum arabic, tragacanth or combinations thereof. Polyhydric alcohols include but are not limited to, polyethylene glycol (PEG) or polypropylene glycol.

Vinyl derivatives, polymers and copolymers thereof include, but are not limited to, polyvinylacetate aqueous dispersion (Kollicoat® SR 30D), copolymers of vinyl pyrrolidone, copolymers of polyvinyl alcohol (Kollicoat® IR), polyvinyl alcohol phthalate, polyvinylacetal phthalate, polyvinyl butylate phthalate, polyvinylacetoacetal phthalate, polyvinylpyrrolidone (PVP) or combinations thereof.

Acrylic acid derivatives include, but are not limited to, methacrylic acids, polymethacrylic acids, polyacrylates, especially polymethacrylates like a) copolymer formed from monomers selected from methacrylic acid, methacrylic acid esters, acrylic acid and acrylic acid esters b) copolymer formed from monomers selected from butyl methacrylate, (2-dimethylaminoethyl)methacrylate and methyl methacrylate c) copolymer formed from monomers selected from ethyl acrylate, methyl methacrylate and trimethylammonioethyl methacrylate chloride or d) copolymers of acrylate and methacrylates with/without quarternary ammonium group in combination with sodium carboxymethylcellulose, e.g. those available from Rohm GmbH under the trademark Eudragit® like Eudragit EPO (dimethylaminoethyl methacrylate copolymer; basic butylated methacrylate copolymer), Eudragit RL and RS (trimethylammonioethyl methacrylate copolymer), Eudragit NE30D and Eudragit NE40D (ethylacrylate methymethacrylate copolymer), Eudragit RD 100 (ammoniomethacrylate copolymer with sodium carboxymethylcellulose); or the like or any combinations thereof.

According to the present invention, non-polymeric pharmaceutically acceptable excipients suitable for formulations of the present invention include, but are not limited to, fats, oils, waxes, fatty acids, fatty acid esters, long chain monohydric alcohols and their esters, phospholipids, terpenes or combinations thereof.

Waxes are esters of fatty acids with long chain monohydric alcohols. Natural waxes are often mixtures of such esters, and may also contain hydrocarbons. Waxes employed in the present invention include, but are not limited to, natural waxes, such as animal waxes, vegetable waxes, and petroleum waxes (i.e., paraffin waxes, microcrystalline waxes, petrolatum waxes, mineral waxes), and synthetic waxes. Specific examples include but are not limited to spermaceti wax, carnauba wax, Japan wax, bayberry wax, flax wax, beeswax, yellow wax, Chinese wax, shellac wax, lanolin wax, sugarcane wax, candelilla wax, castor wax paraffin wax, microcrystalline wax, petrolatum wax, carbowax, and the like, and mixtures thereof.

Waxes are also monoglyceryl esters, diglyceryl esters, or triglyceryl esters (glycerides) and derivatives thereof formed from a fatty acid having from about 10 to about 22 carbon atoms and glycerol, wherein one or more of the hydroxyl groups of glycerol are substituted by a fatty acid. Glycerides employed in the present invention include, but are not limited to, glyceryl monostearate, glyceryl distearate, glyceryl tristearate, glyceryl dipalmitate, glyceryl tripalmitate, glyceryl monopalmitate, glyceryl palmitostearate, glyceryl dilaurate, glyceryl trilaurate, glyceryl monolaurate, glyceryl didocosanoate, glyceryl tridocosanoate, glyceryl monodocosanoate, glyceryl monocaproate, glyceryl dicaproate, glyceryl tricaproate, glyceryl monomyristate, glyceryl dimyristate, glyceryl trimyristate, glyceryl monodecenoate, glyceryl didecenoate, glyceryl tridecenoate, glyceryl behenate, polyglyceryl diisostearate, lauroyl macrogolglycerides, oleoyl macrogolglycerides, stearoyl macrogolglycerides, and combinations thereof.

Fatty acids employed in the present invention include, but are not limited to, hydrogenated palm kernel oil, hydrogenated peanut oil, hydrogenated palm oil, hydrogenated rapeseed oil, hydrogenated rice bran oil, hydrogenated soybean oil, hydrogenated sunflower oil, hydrogenated castor oil, hydrogenated cottonseed oil, and mixtures thereof. Other fatty acids include, but are not limited to, decenoic acid, docosanoic acid, stearic acid, palmitic acid, lauric acid, myristic acid, and the like, and mixtures thereof. In one embodiment the fatty acids employed include, but not limited to, hydrogenated palm oil, hydrogenated castor oil, stearic acid, hydrogenated cottonseed oil, palmitic acid, and mixtures thereof.

Long chain monohydric alcohols employed in the present invention include, but are not limited to, cetyl alcohol, and stearyl alcohol and mixtures thereof.

In one embodiment, the non-polymeric pharmaceutically acceptable excipients employed in the oral pharmaceutical formulations of influenza antiviral of the present invention include, but are not limited to, Cutina® (hydrogenated castor oil), Hydrobase® (hydrogenated soybean oil), Castorwax® (hydrogenated castor oil), Croduret® (hydrogenated castor oil), Carbowax®, Compritol® (glyceryl behenate), Sterotex® (hydrogenated cottonseed oil), Lubritab® (hydrogenated cottonseed oil), Apifil® (wax yellow), Akofine® (hydrogenated cottonseed oil), Softisan® (hydrogenated palm oil), Hydrocote® (hydrogenated soybean oil), Corona® (Lanolin), Gelucire® (macrogolglycerides Lauriques), Precirol® (glyceryl palmitostearate), Emulcire™ (cetyl alcohol), Plurol® diisostearique (polyglyceryl diisostearate), Geleol® (glyceryl stearate),and mixtures thereof.

In another embodiment, lipids or waxes can also be employed in the form of an aqueous dispersion stabilized by surfactants and suitable stabilizers.

The active ingredient is physically mixed or blended with these polymeric or non-polymeric pharmaceutically acceptable excipients or is partially or completely coated with these excipients by any of the techniques known in the art including, but not limited to, microencapsulation, hot melt granulation, melt extrusion, fluid bed coating, wet granulation, spray drying, dry granulation or roll compaction.

In one embodiment, the polymeric or non-polymeric pharmaceutically acceptable excipient is physically mixed with influenza antiviral either alone or along with suitable pharmaceutically acceptable excipients. The polymeric or non-polymeric pharmaceutically acceptable excipient is applied alone or in combination with other suitable pharmaceutical excipients, to influenza antiviral, in the form of, but not limited to, powder, granules, beads, pellets, minitablets or the like to achieve the desired taste-masking.

In another embodiment, the bitter taste of influenza antiviral is masked by using taste-masking agents such as, but not limited to, cyclodextrins, ion exchange resins or carbomers or derivatives thereof.

In one embodiment, taste-masking agent employed in the formulation of the present invention is cyclodextrin or a derivative thereof. In another embodiment, the bitter taste of influenza antiviral is masked by complexation with cyclodextrins or derivatives thereof. Cyclodextrins are cyclic oligosaccharides formed from α-(1,4)-linked D-glucopyranose units. α, β and γ-cyclodextrins consist of six, seven and eight units respectively. Cyclodextrin makes an inclusion complex with the influenza antiviral molecule by acting as a hydrophobic host cavity. Suitable cyclodextrins for use in the formulation of the present invention include, but are not limited to, α, β and γ-cyclodextrins, or alkylated, hydroxyalkylated, esterified, glycosylated or substituted derivatives thereof, such as (2,6-di-o-methyl)-β-cyclodextrin (DIMEB) (dimethyl-β-cyclodextrin), randomly methylated-β-cyclodextrin (RAMEB), and hydroxypropyl-β-cyclodextrin (HPβCD), hydroxyethyl-β-cyclodextrin, dihydroxypropyl-β-cyclodextrin, trimethyl-β-cyclodextrin, hydroxymethyl-β-cyclodextrin, β-cyclodextrin sulfate, β-cyclodextrin sulfonate, methyl-β-cyclodextrin, sulfobutyl ether cyclodextrin (SBE-CD) (β-cyclodextrin sulfobutyl ether), glucosyl-α-cyclodextrin, glucosyl-β-cyclodextrin, diglucosyl-β-cyclodextrin, maltosyl-γ-cyclodextrin, maltosyl-γ-cyclodextrin, maltosyl-γ-cyclodextrin, maltotriosyl-βcyclodextrin, maltotriosyl-γ-cyclodextrin, dimaltosyl-β-cyclodextrin and mixtures thereof such as maltosylβ-cyclodextrin/dimaltosyl-β-cyclodextrin. The complex of active with cyclodextrin can be prepared by various methods such as solution method, co-precipitation method, co-evaporation/solid dispersion method, melting method neutralization method, slurry method, spray drying kneading method, and grinding method. In one embodiment a physical mixture of influenza antiviral and cyclodextrin or a derivative thereof is employed in the composition of the present invention. In one embodiment, the compositions of the present invention comprise influenza antiviral and cyclodextrin or a derivative thereof in an uncomplexed form along with suitable pharmaceutically acceptable excipients.

In one embodiment, the taste-masking agent employed in the formulations of the present invention is an ion exchange resin. In another embodiment, the bitter taste of influenza antiviral is masked by complexing with an ion-exchange resin. Ion-exchange resins are solid and suitably insoluble high molecular weight polyelectrolytes that can exchange their mobile ions of equal charge with the surrounding medium and are not absorbed by the body. The resulting ion exchange is reversible and stoichiometric with the displacement of one ionic species by another. The drug-resin complexes effectively mask the taste of a bitter or unpleasant tasting drug within the matrix of the ion-exchange material. Appropriate selection of the ion-exchange resin is important so that the drug is not released in the mouth, leading to perception of the bitter taste of the drug. The present invention provides a taste-masked influenza antiviral wherein taste-masking is achieved by reversibly binding the active compound onto an ion-exchange resin, wherein the polymeric matrix of the ion-exchange resin has functional groups including, but not limited to, anionic groups, e.g., weakly acidic-carboxylic, esteric and phosphonic; strongly acidic-sulfonic and cationic groups, e.g., weakly basic-tertiary amine; strongly basic-quaternary amine. Additionally suitable polymeric matrices include copolymers of acrylic and substituted acrylic acids; styrene and styrene derivatives; cellulose esters; vinyl and substituted vinyl esters; and polysulfonic acids and polysulfonic acid esters. An ion-exchange resin having the polymeric matrix with an anionic functional group is a cation exchange resin and that having a cationic functional group is an anionic exchange resin. The mobile or exchangeable moieties, depending on the type of resin used, includes, but is not limited to, sodium, hydrogen, potassium, chloride or the like.

In yet another embodiment of the present invention, a cationic exchange resin is used as a taste-masking agent to mask the bitter taste of influenza antivirals. Non-limiting examples of suitable cation exchange resin that may be employed include Amberlite® IRP64 (porous copolymer of methacrylic acid and divinylbenzene), Amberlite® IRP69 (sulfonated copolymer of styrene and divinylbenzene), Amberlite® IRP88 (cross linked polymer of methacrylic acid and divinylbenzene), DOWEX® RTM. resins (strong cationic exchangers based upon polystyrenesulphonic acid with variable crosslinking (1-12% divinylbenzene)), Tulsion® 335—(Polacrilex/{Polacirilex S), Tulsion® 339 (Polacrilin potassium USP), Tulsion® 344 (Sodium polystyrene sulfonate BP), Indion® 204 (crosslinked polyacrylic acid), Indion® 214 (crosslinked polyacrylic acid), Indion® 234 (crosslinked polyacrylic acid), Indion® 234S (crosslinked polyacrylic acid), Indion® 294 (crosslinked polyacrylic acid), Purolite® C115 HMR (carboxylic acid functional group), Purolite® C115 E (carboxylic acid functional group), Purolite® C100 HMR (sulfonic acid functional group), Purolite® 100 MR (sulfonic acid functional group) or cation exchange resins having phosphonic functional groups and the like or any combinations thereof. In one embodiment, ion exchange resin can be used for complexation with influenza antiviral in a ratio of active to resin of about 1:0.1 to about 1:20. These drug resinates can be prepared by methods such as, but not limiting to, blending, kneading, grinding, sieving, filling, compressing, lyophilization, spray-drying, fluid-bed drying or centrifugal granulation.

In another embodiment, the taste-masking agent employed in the formulations of the present invention is a carbomer or a derivative thereof. In yet another embodiment, the bitter taste of influenza antiviral is masked by complexing with carbomers or derivatives thereof. Influenza antivirals can be taste-masked by carbomers such as carbomer 934, carbomer 971, carbomer 974 or the like, wherein the complex is held together by ionic bonding and gel properties of the carbomer, providing stable and palatable formulations. These complexes can be prepared by mixing, blending or slurrying influenza antiviral and carbomer together to allow the desired complex formation.

In a further embodiment, the bitter taste of influenza antivirals is masked by using as taste-masking agent adsorbents that form adsorbates with the antiviral. Adsorbates can be formed by adsorbing or partially or significantly blending influenza antivirals with an adsorbent including, but not limited to, activated granular carbon, active aluminum, clay, bentonite, kaolite, zeolite, sodium alginate, magnesium aluminium silicate, silica gel, or activated charcoal and mixtures thereof. These adsorbent materials surround the drug particles by forming a physical bond, by Van der Waals interactions and hydrogen bonding so that the bitter taste of the drug is not perceived. The adsorbate of influenza antiviral can be formed by mixing or blending the active with the adsorbent in high or moderate shear mixers like planetary mixer or rapid mixer granulator. Alternatively, adsorbate can be formed by wet granulation involving the adsorbent and influenza antiviral in any conventional granulation equipment.

In a yet another embodiment, a sugar substitute is employed as a taste-masking agent that masks the bitter taste of influenza antiviral by salt formation. The bitter taste of influenza antiviral is masked using equimolar amounts of sugar substitutes such as but not limited to cyclamate, saccharin, acesulfame or a mixture of at least two of the sugar substitutes by salt formation. Such a treatment results in the formation of taste-masked influenza antiviral salts that have the desired taste and also improved patient compliance. Such a taste-masked salt can be incorporated in pharmaceutical formulations for oral administration.

In one embodiment the amount of taste-masking agent employed for the preparation of taste-masked influenza antiviral formulations of the present invention can be in the range from about 1% to about 95% by weight of the formulation. In another embodiment the amount of taste-masking agent employed for the preparation of taste-masked influenza antiviral formulations of the present invention can be in the range from about 2% to about 75% by weight of the formulation. In another embodiment the amount of taste-masking agent employed for the preparation of taste-masked influenza antiviral formulations of the present invention can be in the range from about 5% to about 60% by weight of the formulation.

In one embodiment the taste masked influenza antiviral of the present invention in the form of, but not limited to, powder, granules, pellets, beads, minitablets or the like can be administered as such or are suitable for incorporation into various oral dosage forms including, but not limited to, orally disintegrating, dispersible, chewable or effervescent tablets, sprinkle granules, quick melt wafers, lozenge, dry suspensions or syrups for reconstitution, chewing gum or the like. These oral formulations may contain from about 5% to about 95% of taste-masked influenza antiviral. In one embodiment the taste masked influenza antiviral formulation for oral administration can be provided in the form of various dosage forms, such as but not limited to orally disintegrating, dispersible, chewable or effervescent tablets, sprinkle granules, quick melt wafers, lozenge, dry suspensions or syrups for reconstitution, chewing gum or the like. The taste-masked formulations may be so designed that the in vitro dissolution and bioavailability of the influenza antiviral is not compromised.

The taste-masked influenza antiviral formulations of the present invention may further comprise at least one pharmaceutically acceptable excipient including, but not limited to, binder, disintegrant, superdisintegrant, diluent, salivating agent, surfactant, flavor, sweetener, colorant, souring agent, viscolizer, glidant, lubricant, solubilizer, stabilizer or the like, depending on the dosage form.

In one embodiment, the taste-masked influenza antiviral formulation of the present invention is in the form of dispersible tablets. Dispersible tablet refers to a tablet which disperses in aqueous phase, e.g., in water before administration. A water-dispersible tablet, according to the British Pharmacopoeia and European Pharmacopoeia, should meet the requirements of the test for dispersible tablets as regards dispersion time (<3 minutes) and dispersion quality (i.e. to pass through a 710 μm sieve).

The dispersible tablet formulations comprising influenza antiviral and at least one taste-masking agent further comprises at least one pharmaceutically acceptable excipient including, but not limited to, one or more binders, disintegrants, superdisintegrants, diluents, salivating agents, surfactants, flavors, sweeteners, colorants, diluents, souring agents, viscolizers, glidants or lubricants, solubilizers, or stabilizers.

Examples of suitable binders include, but are not limited to, starch, pregelatinized starch, polyvinyl pyrrolidone (PVP), Copovidone, cellulose derivatives, such as hydroxypropylmethyl cellulose (HPMC), hydroxypropyl cellulose (HPC) and carboxymethyl cellulose (CMC) and their salts. Examples of suitable diluents include, but are not limited to, starch, microcrystalline cellulose, lactose, xylitol, mannitol, maltose, polyols, fructose, guar gum, sorbitol, magnesium hydroxide, dicalcium phosphate, and the like or any combinations thereof. Examples of superdisintegrants include, but are not limited to, natural, modified or pregelatinized starch, crospovidone, croscarmellose sodium, sodium starch glycolate, low-substituted hydroxypropyl cellulose as well as effervescent disintegrating systems. Further, the disintegrants include, but are not limited to, crosspovidone, calcium silicate and starch. The amount of superdisintegrant employed in the formulation is about 2% to about 30% by weight of the said dosage form. Examples of lubricant include, but are not limited to, magnesium stearate, calcium stearate, stearic acid, talc, and sodium stearyl fumarate. The tablet formulation of the invention may also include a glidant such as, but not limited to, colloidal silica, silica gel, precipitated silica, or combinations thereof. The said formulations may also include salivating agents such as, but not limited to, micronised polyethylene glycol, sodium chloride or precipitated micronised silica to improve the disintegration properties of the said formulations. Examples of solubilizers include, but are not limited, to cetostearyl alcohol, cholesterol, diethanolamine, ethyl oleate, ethylene glycol palmitostearate, glycerin, glyceryl monostearate, isopropyl myristate, lecithin, medium-chain glyceride, monoethanolamine, oleic acid, propylene glycol, polyoxyethylene alkyl ether, polyoxyethylene castor oil glycoside, polyethylene sorbitan fatty acid ester, polyoxyethylene stearate, propylene glycol alginate, sorbitan fatty acid ester, stearic acid, sunflower oil, triethanolmine, and mixtures thereof. The formulations of the present invention may also include stabilizers such as, but not limited to, benzoic acid, sodium benzoate, citric acid, and the like. Examples of surfactants include, but are not limited to, sodium docusate, glyceryl monooleate, polyethylene alkyl ether, polyoxyethylene sorbitan fatty acid ester, sodium lauryl sulfate, sorbic acid, sorbitan fatty acid ester, and mixtures thereof. Souring agents include, but are not limited to, monosodium fumarate and/or citric acid.

The formulations of the present invention may optionally include viscolizer agents such as, for example, polyalkylene oxides; polyols; starch and starch-based polymers; chitosan; polysaccharide gums; polyethylene oxide, hydroxypropyl cellulose, hydroxypropyl methyl cellulose, hydroxyethyl cellulose, sodium carboxy methylcellulose, calcium carboxymethyl cellulose, methyl cellulose, polyacrylic acid, gum acacia, gum tragacanth, xanthan gum, guar gum and polyvinyl alcohol and copolymers and mixtures thereof. Examples of viscolizers which can be used include, but are not limited to, polyalkylene oxides such as polyethylene oxide; cellulose ethers such as hydroxyethyl cellulose, hydroxypropylcellulose, hydroxypropyl methyl cellulose, methyl cellulose, ethyl cellulose, sodium carboxy methylcellulose, calcium carboxymethyl cellulose, microcrystalline cellulose; gums such as gum arabic alginates, agar, guar gum, locust bean, carrageenan, tara, gum arabic, tragacanth, pectin, xanthan, gellan, maltodextrin, galactomannan, pusstulan, laminarin, scleroglucan, gum arabic, inulin, karaya, whelan; polyols such as dipropylene glycol, polypropylene glycol, propylene glycol, polyethylene glycol (PEG), sorbitol and glycerol; carbopol, starch and starch-based polymers such as pregelatinized starch, acrylic acid and methacrylic acid polymers, and esters thereof, maleic anhydride polymers; polymaleic acid; poly(acrylamides); poly(olefinic alcohol)s; poly(N-vinyl lactams); polyoxyethylated saccharides; polyoxazolines; polyvinylamines; polyvinylacetates; polyimines; povidone vinylpyrrolidone/vinyl acetate copolymer and polyvinyl acetate, mixture of polyvinyl acetate and polyvinylpyrrolidone, chitin, cyclodextrin, gelatin, chitosan, and combinations thereof. In one embodiment viscolizers are hydroxypropyl cellulose, hydroxypropyl methylcellulose, hydroxyethyl cellulose, polyethylene oxide, sodium carboxy methylcellulose, microcrystalline cellulose, guar gum, xanthan gum, alginates and combinations thereof. In one embodiment the weight percent of the viscolizer in the dispersible tablet dosage form is about 2 to about 75 weight percent. In another embodiment the weight percent of the viscolizer in the dispersible tablet dosage form is about 10 to about 70 weight percent. In yet another embodiment the weight percent of the viscolizer in the dispersible tablet dosage form is about 5 to about 50 weight percent. The viscolizers act to control sedimentation rate of dispersed influenza antiviral thereby producing homogeneous dispersions when the dispersible tablets are dispersed in water before administration thus ensuring substantially uniform dosing. They rapidly generate viscosity when the dispersible tablets come in contact with water, and a homogenous suspension is formed, which can be easily swallowed by children and the elderly, with minimal effect of the release properties of the biologically active ingredient.

Further, the taste-masked influenza antiviral formulations of the present invention also comprise at least one sweetener such as, but not limited to, aspartame, stevia extract, glycyrrhiza, saccharine, saccharine sodium, acesulfame, sucralose and dipotassium glycyrrhizinate; and/or one or more flavors, e.g., mint flavour, orange flavour, lemon flavors, strawberry aroma, vanilla flavour, raspberry aroma, cherry flavor, tutty frutty flavor, magnasweet 135, key lime flavor, grape flavor, trusil art 511815, and fruit extracts.

In another embodiment, taste-masked influenza antiviral formulation is in the form of an effervescent tablet. Effervescent tablets are intended to be dissolved or dispersed in water before administration and generally contain acid substances and carbonates or bicarbonates, which react rapidly in the presence of water releasing carbon dioxide. These tablets comprise various pharmaceutically acceptable excipients as have been discussed under dispersible tablets. The effervescent tablets can comprise effervescent couples such as, but not limited to, thermolabile gas generating agents such as sodium bicarbonate, sodium glycine carbonate, potassium bicarbonate, ammonium bicarbonate, sodium bisulfite, sodium metabisulfite, and an acid source such as citric acid, maleic acid or tartaric acid.

In yet another embodiment, the taste-masked influenza antiviral formulation of the present invention is in the form of an orally disintegrating tablet. Orally disintegrating tablets (ODTs) disintegrate/dissolve in the mouth rapidly without administering extra water, providing the convenience of a tablet formulation while allowing the ease of swallowing provided by a liquid formulation.

The orally disintegrating tablets comprising influenza antiviral can further comprise as filler, binder or disintegrant, a directly compressible coprocessed excipient. PCT Application WO2007052289 describes directly compressible coprocessed excipient comprising of at least one water soluble excipient and water insoluble inorganic excipient such as calcium silicate. The water soluble carbohydrate can be a monosaccharide, disaccharide, oligosaccharide or polysaccharide. Examples of carbohydrates include, but are not limited to, monosaccharides such as sorbitol, glucose, dextrose, fructose, maltose or xylitol, disaccharides such as sucrose, trehalose, lactose, glucose, galactose or mannitol, and oligosaccharides and polysaccharides such as dextrates and maltodextrins. The water soluble and water insoluble excipients in the directly compressible coprocessed excipient can be in a ratio of water-soluble excipient to water insoluble excipient of from about 50:1 to about 1:50. In one embodiment of the present invention, this ratio is about 30:1 to about 1:30. In a further embodiment of the present invention, this ratio is from about 20:1 to about 1:20. The amount of directly compressible coprocessed excipient employed in the orally disintegrating tablet formulations comprising influenza antiviral and at least one taste-masking agent is about 5% to about 95% by weight of the dosage form.

The formulations of the present invention in the form of orally disintegrating tablets may include, in addition to the influenza antiviral, at least one taste-masking agent and directly compressible coprocessed excipient, one or more pharmaceutically acceptable excipients as discussed under dispersible tablets above.

The orally disintegrating tablet formulations can be prepared by any of the known non limiting techniques such as freeze-drying, molding and sublimation, compression, cotton candy process, mass extrusion, etc or with use of specialized excipients such as effervescent couple, highly micronized agents, coprocessed excipients or the like.

The orally disintegrating tablet formulations based on taste-masked influenza antiviral dissolve or disintegrate in the oral cavity, preferably within about 60 seconds.

In one embodiment of the present invention, the taste-masked influenza antiviral formulation is in the form of bite-dispersion tablets. Bite-dispersion tablets are meant to be taken without water and disperse easily, and quickly, after a gentle bite when taken orally. These tablets comprise various pharmaceutically acceptable excipients as have been discussed under dispersible tablets in addition to excipients which may be specifically employed for bite-dispersion tablets.

In another embodiment of the present invention, the taste-masked influenza antiviral formulation is in the form of chewable tablets. Chewable tablets are taken slowly by chewing or sucking in the mouth, and enable taste-masked active contained therein to be orally administered without water. These chewable tablets comprise various pharmaceutically acceptable excipients as have been discussed under dispersible tablets in addition to excipients which may be specifically employed for chewable tablets.

The terms “tablet”, “tablet composition” and “tablet formulation” are used synonymously within the context of the present invention. These terms should be construed to include a compacted or compressed powder composition obtained by compressing or otherwise forming the composition to form a solid having a defined shape. Tablets in accordance with the invention may be manufactured using conventional techniques of common tableting methods known in the art such as direct compression, wet granulation, dry granulation and extrusion/melt granulation. In one embodiment, the process is direct compression which involves compression of taste-masked drug-excipient blend after mixing them for a definite time period. The tablet may vary in shape such as oval, triangle, almond, peanut, parallelogram, round, pentagonal, hexagonal, and trapezoidal. The preferred shapes are round, oval and parallelogram forms.

In another embodiment of the present invention, the taste-masked influenza antiviral formulation can be incorporated in sprinkle granules, quick melt wafers, lozenge, dry suspensions or syrups for reconstitution, chewing gum or the like.

The various dosage forms as described in the present invention comprising influenza antiviral, at least one taste-masking agent and at least one pharmaceutically acceptable excipient are preferably immediate release dosage forms that release influenza antiviral instantly upon reaching either stomach or intestine. The formulations disclosed in present invention can also be adapted to develop a formulation wherein influenza antiviral is released in a controlled manner over a period of time, for example, from about 2 to about 24 hours. In such a formulation, influenza antiviral is treated with polymeric, non-polymeric pharmaceutically acceptable excipients described above or any combinations thereof. The amount of such polymeric or non-polymeric excipients not only ensures masking of the objectionable taste of the active but also controls the release of influenza antiviral.

In another embodiment of the present invention, pellets or granules or the like of influenza antiviral are prepared comprising at least one release retardant in combination with one or more pharmaceutically acceptable excipients. Suitable release retardants, as discussed above can be polymeric or non-polymeric pharmaceutically acceptable excipients or agents and include, but are not limited to, cellulose ethers, such as hydroxypropylmethylcellulose (HPMC), hydroxypropylcellulose (HPC), hydroxyethylcellulose, ethyl cellulose and carboxymethylcellulose sodium; polysaccharides, such as carageenan, guar gum, xanthan gum, tragacanth and ceratonia; polymethacrylates, such as copolymers of acrylic and methacrylic acid esters containing quarternary ammonium groups; cellulose esters, such as cellulose acetate; acrylic acid polymers, such as carbomers; waxes, such as hydrogenated castor oil, hydrogenated vegetable oil, carnauba wax and microcrystalline wax; alginates, such as alginic acid and sodium alginate; and fatty acid derivatives, such as glyceryl monostearate and glyceryl palmitostearate. These pellets or granules or the like can be further coated using excipients described above in order to achieve taste-masking and controlled release of influenza antiviral. The amount of release retardant in the formulation is from about 1 to 90% by weight of the dosage form. In one embodiment, the amount of release retardant in the formulation is about 5 to 80% by weight of the dosage form.

The present invention discloses a process for preparing taste-masked pharmaceutical formulation of influenza antiviral comprising:
(a) coating an influenza antiviral with at least one taste-masking agent to form a taste-masked influenza antiviral;
(b) blending the taste-masked influenza antiviral of step (a) with other excipients, except lubricant, to form a uniform powder mix;
(c) lubricating the powder mix of step (b); and
(d) compressing the powder mix of step (c) into a dispersible tablet composition,
   wherein at least one taste-masking agent is a polymeric pharmaceutically acceptable excipient, a non-polymeric pharmaceutically acceptable excipient, or a combination thereof.

The present invention further discloses a process for preparing a taste-masked pharmaceutical formulation of an influenza antiviral comprising:
(a) physically mixing an influenza antiviral with at least one taste-masking agent;
(b) blending the mix of step (a) with other excipients, except lubricant, to form a uniform powder mix;
(c) lubricating the powder mix of step (b); and
(d) compressing the powder mix of step (c) into a dispersible tablet composition,
   wherein at least one taste-masking agent is a polymeric pharmaceutically acceptable excipient, a non-polymeric pharmaceutically acceptable excipient, or a combination thereof.

The present invention also discloses a process for preparing a taste-masked pharmaceutical formulation of influenza antiviral comprising:
(a) complexing an influenza antiviral with at least one taste-masking agent to form a taste-masked influenza antiviral;
(b) blending the taste-masked influenza antiviral of step (a) with other excipients, except lubricant, to
form a uniform powder mix;
(c) lubricating the powder mix of step (b); and
(d) compressing the powder mix of step (c) into a dispersible tablet composition,
   wherein at least one taste-masking agent is an ion exchange resin, a carbomer, a cyclodextrin or a derivative thereof.

The present invention also discloses a process for preparing a taste-masked pharmaceutical formulation of influenza antiviral comprising:
(a) adsorbing an influenza antiviral with at least one taste-masking agent to form a taste-masked influenza antiviral;
(b) blending the taste-masked influenza antiviral of step (a) with other excipients, except lubricant, to form a uniform powder mix;
(c) lubricating the powder mix of step (b); and
(d) compressing the powder mix of step (c) into a dispersible tablet composition,
   wherein at least one taste-masking agent is an adsorbent.

The present invention further provides a method of prevention and/or treatment of any confirmed, probable or suspected influenza virus infection by administering to a patient in need thereof formulation of the present invention comprising an influenza antiviral, at least one taste-masking agent and at least one pharmaceutically acceptable excipient. In another embodiment, there is provided use of the formulation of the present invention comprising an influenza antiviral, at least one taste-masking agent and at least one pharmaceutically acceptable excipient for the manufacture of a medicament for the prevention and/or treatment of any confirmed, probable or suspected influenza virus infection.

In a still further embodiment of the present invention the taste-masked influenza antiviral formulations of the present invention may be adapted to deliver one or more active agents in addition to influenza antivirals. Complications of influenza can include bacterial infections, viral pneumonia, and cardiac and other organ system abnormalities. People with chronic medical conditions may have increased risk of complications when they get influenza. Many other diseases, including serious infections such as rapidly progressive bloodstream infections, may start with symptoms that resemble influenza and may need to be considered in treatment decisions. Therefore, the active agent that may be combined with the influenza antiviral includes, but is not limited to, nelfinavir, abacavir, acemetacin, acetoniode, acetyl salicylic acid, aciclovir, acrivastine, acyclovir, adefovir, alclofenac, alminoprofen, amastatin, amprenavir, antipain, aprotinin, arbidol, astemizole, atazanavir, atorvastatin, atripla, azapropazone, azelastine, benorylate, benoxaprofen, betastatin, boceprevir, bucloxic acid, budesonide, caffeine, carprofen, celecoxib, cerivastatin, cetirizine, choline magnesium trisalicylate, cidofovir, clidanac, clopinac, combivir, darunavir, delavirdine, deracoxib, desloratadine, diclofenac, didanosine, diflunisal, docosanol, droxicam, ebastine, edoxudine, efavirenz, elastatinal, emtricitabine, enfuvirtide, entecavir, epinastine, etodolac, etoricoxib, famciclovir, fenbufen, fenclofenec, fenoprofen, fentiazac, feprazone, fexofenadine, floctafenine, flufenamic acid, flufenisal, fluprofen, flurbiprofen, fluticosone propionate, fluvastatin, fomivirsen, fosamprenavir, foscarnet, fosfonet, furofenac, ganciclovir, ibacitabine, ibufenac, ibuprofen, idoxuridine, imiquimod, imunovir, indinavir, indomethacin, indoprofen, isoxepac, isoxicam, ketoprofen, ketorolac, lamivudine, leupeptin, licofelone, lopinavir, lortadine, lovastatin, loviride, lumiracoxib, maraviroc, meclofenamic acid, mefenamic acid, miroprofen, mizolastine, mometasone furoate mononhydrate, moroxydine, nabumetone, naproxen, nelfinavir, nevirapine, nexavir, niflumic acid, norastemizole, oxaprozin, oxipinac, oxyphenbutazone, parecoxib, penciclovir, pepstatin, peramivir, phenylbutazone, piprofen, piroxicam, pirprofen, pleconaril, prapoprofen, pravastatin, proglumetacin, prometazine, raltegravir, ribavirin, ritonavir, rofecoxib, saquinavir, simvastatin, stavudine, sudoxicam, sulindac, suprofen, tenofovir, tenoxicam, terfenadine, tepoxalin, thiophosphonoformic acid, tiaprofenic acid, tiopinac, tioxaprofen, tipranavir, tolfenamic acid, tolmetin, triamcinalone, trifluridine, trizivir, tromantadine, truvada, valaciclovir, valdecoxib, valganciclovir, vicriviroc, vidarabine, viramidine, zalcitabine, zidometacin, zidovudine, zomepirac, ceftriaxone, cefotaxime, vancomycin, meropenem, cefepime, ceftazidime, cefuroxime, nalcillin, oxacillin, ampicillin, ticarcillin, ticarcillin/clavulinic acid, ampicillin/sulbactam, trimethoprim-sulfamethoxazole, clindamycin, synercid, amoxicillin, amoxicillin/clavulinic acid, cefuroxime, trimethoprim/sulfamethoxazole, azithromycin, dicloxacillin, ciprofloxacin, levofloxacin, cefixime, cetpodoxime, loracarbef, cefadroxil, cefabutin, cefdinir, cephradine or the like or mixtures thereof.

While the present invention has been described in terms of its specific embodiments, certain modifications and equivalents will be apparent to those skilled in the art and are intended to be included within the scope of the present invention. Details of the present invention, including its objects and advantages, are provided in the non-limiting exemplary illustrations below.

### EXAMPLES

### Example 1

### Dispersible Tablets of Taste-Masked Oseltamivir Phosphate

Oseltamivir phosphate 59.1 mg (equivalent to 45 mg oseltamivir base) was layered on 100 mg non-pareil beads and these drug-loaded beads were further coated with a combination of ethyl cellulose and hydroxypropyl methylcellulose (20:80) to a weight gain of about 20%.

TABLE 1
Preparation of dispersible tablet formulation

| Ingredients | mg/unit |
|---|---|
| Taste-masked oseltamivir phosphate granules | 190.92 |
| equivalent to 45 mg oseltamivir base | |
| Microcrystalline cellulose, USP | 100.08 |
| Hydroxyethyl cellulose, USP | 65 |
| Crospovidone, USP/NF | 55 |
| Hydroxy propyl cellulose, USP | 18 |
| Mannitol, USP | 25 |
| Copovidone, Ph. Eur. | 14 |
| Aspartame, USP | 20 |
| Vanilla flavor, USP | 5 |
| Colloidal silicon dioxide, USP | 4 |
| Magnesium stearate, USP | 3 |
| Total | 500 |

Procedure: All the ingredients were sifted and blended with taste-masked oseltamivir phosphate. The blend was lubricated and compressed as dispersible tablets. The dispersible tablets had excellent palatability and passed the disintegration and dispersibility tests as per Ph. Eur. (4^{th} Edition). The dispersion produced in water with the tablets showed desired rate of sedimentation, with not more than 50% of the active settling in 5 minutes.

### Example 2

### Dispersible Tablets of Taste-Masked Oseltamivir Phosphate

Oseltamivir phosphate 19.7 mg (equivalent to 15 mg oseltamivir base) was mixed with beta-cyclodextrin 54.5 mg in a ball mill (1:1 molar ratio) for about 5 hours and this taste-masked oseltamivir phosphate was then incorporated into dispersible tablet formulation.

TABLE 2
Preparation of dispersible tablet formulation

| Ingredients | mg/unit |
|---|---|
| Taste masked oseltamivir phosphate | 74.2 |
| equivalent to 15 mg oseltamivir base | |
| Microcrystalline cellulose, USP | 72.8 |
| Hydroxy ethyl cellulose, USP | 40 |
| Crospovidone, USP/NF | 30 |
| Hydroxy propyl cellulose, USP | 15 |
| Mannitol, USP | 25 |
| Copovidone, Ph. Eur. | 12 |
| Aspartame, USP | 20 |
| Vanilla flavor, USP | 5 |
| Colloidal silicon dioxide, USP | 3 |
| Magnesium stearate, USP | 3 |
| Total | 300 |

Procedure: All the ingredients were sifted and blended with taste-masked oseltamivir phosphate. The blend was lubricated and compressed as dispersible tablets. The dispersible tablets had excellent palatability and passed the disintegration and dispersibility tests as per Ph. Eur. (4^{th} Edition).

### Example 3

### Dispersible Tablets of Taste-Masked Oseltamivir Phosphate

TABLE 3
Pelletization of oseltamivir phosphate

| Ingredients | mg/unit |
|---|---|
| Oseltamivir phosphate equivalent | 78.8 |
| to 60 mg oseltamivir base | |
| Microcrystalline cellulose, USP | 33.2 |
| Crospovidone, USP/NF | 3 |
| Acesulfame K, Ph. Eur. | 5 |
| Copovidone, Ph. Eur. | 5 |
| Total | 125 |

Procedure: All the ingredients were blended and copovidone solution in water was added to the blend to get wet mass. The mass was extrudated, spheronized and dried. These pellets were then further coated with the coating system of Table 4.

TABLE 4
Preparation of aqueous wax coating solution

| Ingredients | % w/w |
|---|---|
| Hydrogenated vegetable oil, USPNF | 7.5 |
| Glyceryl Mono- & dicaprate, USPNF | 2 |
| Hydroxy propyl methyl cellulose, USP | 2.25 |
| Talc, USP | 1 |
| D.M water | q.s. |

Procedure: Coating emulsion was prepared by melting hydrogenated vegetable oil in water bath and adding glyceryl mono- & dicaprate in molten wax. Hydroxy propyl methyl cellulose was dissolved in water and this aqueous phase was added to oily phase. Talc was added to the emulsion, homogenized well and cooled to room temperature.

Oseltamivir phosphate pellets were coated with the coating emulsion of table 4 to a weight gain of 30% using the bottom spray assembly.

TABLE 5
Preparation of dispersible tablet formulation

| Ingredients | mg/unit |
|---|---|
| Taste masked oseltamivir phosphate pellets | 162.5 |
| equivalent to 60 mg oseltamivir base | |
| Microcrystalline cellulose, USP | 185.5 |
| Crospovidone, USP/NF | 55 |
| Sodium saccharine, USP | 32 |
| Calcium silicate, USP | 16 |
| Hydroxyethyl cellulose, USP/NF | 140 |
| Peppermint flavour | 4 |
| Magnesium stearate, USP | 5 |
| Total | 600 |

Procedure: All the ingredients were sifted and blended with taste-masked oseltamivir phosphate. The blend was lubricated and compressed as dispersible tablets. The dispersible tablets had excellent palatability and passed the disintegration and dispersibility tests as per Ph. Eur. (4^{th} Edition).

### Example 4

### Dispersible Tablets of Taste-Masked Oseltamivir Phosphate

TABLE 6
Preparation of dispersible tablet formulation

| Ingredients | mg/unit |
|---|---|
| Oseltamivir phosphate equivalent to | 39.4 |
| 30 mg oseltamivir base | |
| Stearoyl macrogol glycerides, USP | 80 |
| Microcrystalline cellulose, USP | 135.6 |
| Hydroxy ethyl cellulose, USP | 85 |
| Crospovidone, USP/NF | 40 |
| Hydroxy propyl cellulose, USP | 30 |
| Mannitol, USP | 40 |
| Copovidone, Ph. Eur. | 12 |
| Aspartame, USP | 25 |
| Vanilla flavor, USP | 5 |
| Colloidal silicon dioxide, USP | 4 |
| Magnesium stearate, USP | 4 |
| Total | 500 |

Procedure: Oseltamivir phosphate was melt granulated with stearoyl macrogol glycerides, USP. The granules were then blended with other excipients except lubricant in a blender to get a uniform powder mix. The mass was lubricated and compressed into dispersible tablets. The dispersible tablets had excellent palatability and passed the disintegration and dispersibility tests as per Ph. Eur. (4^{th} Edition).

### Example 5

### Orally Disintegrating Tablets of Taste-Masked Oseltamivir Phosphate

TABLE 7
Preparation of orally disintegrating tablet formulation

| Ingredients | mg/tab |
|---|---|
| Oseltamivir phosphate equivalent to 30 mg | 39.4 |
| oseltamivir base | |
| Basic butylated methacrylate copolymer, Ph. Eur. | 80 |
| Maize Starch, USP | 44 |
| Directly compressible excipient comprising | 165.6 |
| mannitol and calcium silicate | |
| Microcrystalline cellulose, USP | 65 |
| Crospovidone, USP/NF | 28 |
| Aspartame, USP | 20 |
| Peppermint flavor | 3 |
| Magnesium stearate, USP | 2 |
| Colloidal silicon dioxide, USP | 3 |
| Total | 450 |

Procedure: Oseltamivir phosphate was thoroughly dry mixed with basic butylated methacrylate copolymer and then blended with other excipients except lubricant in a blender to get a uniform powder mix. The mix was lubricated and compressed into tablets that had desired taste-masking, friability, disintegration time. The formulation was palatable with pleasant mouth feel.

### Example 6

### Chewable Tablets of Taste-Masked Amantadine Hydrochloride

TABLE 8
Preparation of taste-masked amantadine hydrochloride
granules using non-polymeric taste-masking agent

| Ingredients | mg/unit |
|---|---|
| Amantadine hydrochloride | 100 |
| Glyceryl behenate, USP | 100 |
| Dextrate hydrated, USP | 40 |
| Total | 240 |

Procedure: Weighed quantity of dextrate hydrated was mixed with amantadine hydrochloride and added to molten glyceryl behenate to form a homogenous mixture that was allowed to cool to room temperature and sifted to obtain taste-masked granules.

TABLE 9
Preparation of chewable tablet formulation

| Ingredients | mg/unit |
|---|---|
| Taste masked granules of amantadine hydrochloride | 240 |
| equivalent to 100 mg amantadine hydrochloride | |
| Spray dried mannitol, USP | 175 |
| Maize starch, USP | 55 |
| Lactose monohydrate, USP | 25 |
| Crospovidone, USP/NF | 35 |
| Aspartame, USP | 7 |
| Acesulfame K, Ph. Eur. | 7 |
| Colloidal silicon dioxide, USP | 3 |
| Magnesium stearate, USP | 2 |
| Flavor (Tutty Frutty) | 1 |
| Total | 550 |

Procedure: All the ingredients except colloidal silicon dioxide and magnesium stearate were added to the taste-masked amantadine hydrochloride granules and blended well. The blend was lubricated and compressed into tablets that had desired taste-masking, friability, disintegration time. The formulation was palatable with pleasant mouth feel.

### Example 7

### Chewable Tablets of Taste-Masked Oseltamivir Phosphate

TABLE 10
Preparation of chewable tablet formulation of
oseltamivir phosphate taste-masked using ion exchange resin

| Ingredients | mg/unit |
|---|---|
| Oseltamivir phosphate equivalent to 45 mg oseltamivir base | 59.1 |
| Divinyl Benzene and sulphonic acid copolymer with Na⁺ | 120 |
| function, USPNF (Amberlite IRP 69) | |
| Spray dried mannitol, USP | 150 |
| Microcrystalline cellulose, USP | 85 |
| Aspartame, USP | 22 |
| Sodium starch glycolate, USP | 30 |
| Colloidal silicon dioxide , USP | 6 |
| Orange Flavor | 5.9 |
| Magnesium stearate, USP | 7 |
| Total | 485 |

Procedure: Oseltamivir phosphate was treated with ion exchange resin Amberlite IRP 69. The complex was then blended well with other excipients except the lubricants. Finally, the blend was lubricated and compressed to get a chewable tablet. The tablets had desired taste, mouth feel, friability, disintegration time and in vitro release profile.

### Example 8

### Bite-Dispersion Tablet Formulation of Taste-Masked Oseltamivir Phosphate

TABLE 11
Preparation of melt extruded pellets of oseltamivir phosphate

| Ingredients | mg/unit |
|---|---|
| Oseltamivir phosphate equivalent to 75 mg | 98.5 |
| oseltamivir base | |
| Glyceryl behenate, USP | 100 |
| Dibutyl phthalate, USP | 6.5 |
| Total | 205 |

Procedure: Oseltamivir phosphate pellets suitable for formulation as taste-masked multiparticulates were prepared using the extrusion and spheronization technique. The resultant beads were further coated with the coating system of Table 12 to a weight gain of 10% by weight for additional release rate control and taste-masking.

TABLE 12
Composition of coating system

| Ingredients | % w/w |
|---|---|
| Polyvinyl acetate dispersion 30%, Ph. Eur | 60 |
| Propylene glycol, USP | 1.5 |
| Water | q.s |

TABLE 13
Preparation of bite-dispersion tablet formulation

| Ingredients | mg/tab |
|---|---|
| Taste-masked oseltamivir phosphate pellets | 225.5 |
| equivalent to 75 mg oseltamivir base | |
| Directly compressible excipient comprising | 150 |
| mannitol and calcium silicate | |
| Maize starch, USP | 25 |
| Microcrystalline cellulose, USP | 24 |
| Crospovidone, USP | 10 |
| Aspartame, USP | 12 |
| Colloidal silicon dioxide, USP | 3 |
| Magnesium stearate, USP | 3 |
| Banana Flavor | 2.5 |
| Total | 455 |

Procedure: Taste-masked coated oseltamivir phosphate pellets were mixed with other excipients and compressed into bite dispersion tablets. Tablets with desired taste, mouth feel, friability, disintegration time and in vitro release profile were obtained.

### Example 9

### Chewable Tablets of Rimantadine Hydrochloride

TABLE 14
Wet granulation of rimantadine hydrochloride
using polymeric taste-masking agent

| Ingredients | mg/unit |
|---|---|
| Rimantadine hydrochloride | 100 |
| Basic butylated methacrylate copolymer, Ph. Eur | 70 |
| Microcrystalline cellulose, USP | 50 |
| Aspartame, USP | 18 |
| Croscarmellose sodium, USP/NF | 6 |
| Polyvinylpyrrolidone, USP/NF | 6 |
| Total | 250 |

Procedure: Rimantadine hydrochloride and basic butylated methacrylate copolymer were dry mixed, to which microcrystalline cellulose, aspartame and croscarmellose sodium were added. The mixture was blended well and wet granulated using polyvinyl pyrrolidone to obtain taste-masked rimantadine hydrochloride granules.

TABLE 15
Preparation of chewable tablet formulation
of rimantadine hydrochloride

| Ingredients | mg/unit |
|---|---|
| Taste masked rimantadine hydrochloride granules | 250 |
| equivalent to 100 mg rimantadine hydrochloride | |
| Microcrystalline cellulose, USP | 75 |
| Spray dried mannitol, USP | 100 |
| Maize Starch, USP | 30 |
| Lactose monohydrate , USP | 30 |
| Crospovidone, USP/NF | 36 |
| Acesulfame K, Ph. Eur. | 12 |
| Colloidal silicon dioxide, USP | 3 |
| Magnesium stearate, USP | 3 |
| Flavor (Grape) | 1 |
| Total | 540 |

Procedure: All the ingredients were sifted and blended with taste masked rimantadine hydrochloride. The blend was lubricated and compressed as chewable tablets. Tablets with desired taste, mouth feel, friability, disintegration time and in vitro release profile were obtained.

1. A taste-masked pharmaceutical formulation for oral administration comprising:
(a) at least one influenza antiviral;
(b) at least one taste-masking agent; and
(c) at least one pharmaceutically acceptable excipient.

2. The formulation of claim 1 wherein said influenza antiviral is oseltamivir, zanamivir, peramivir, amantadine or rimantadine in the form of a free base, a pharmaceutically acceptable salt, a prodrug, an active metabolite, a polymorph, a solvate, a hydrate, an enantiomer, an optical isomer, a tautomer or a racemic mixture thereof.

3. (canceled)

4. The formulation of claim 1 wherein said influenza antiviral is oseltamivir phosphate.

5. The formulation of claim 1 wherein said taste-masking agent is a polymeric pharmaceutically acceptable excipient, a non-polymeric pharmaceutically acceptable excipient, an adsorbent, a carbomer, an ion exchange resin, a sugar substitute, a cyclodextrin or a derivative thereof, or a combination thereof.

6. The formulation of claim 5 wherein said polymeric pharmaceutically acceptable excipient is a cellulose derivative, a saccharide or polysaccharide, a polyhydric alcohol, a poly(oxyethylene)-poly(oxypropylene) block copolymer, a vinyl derivative or polymer or copolymer thereof, or a acrylic acid derivative, or a combination thereof.

7. The formulation of claim 6 wherein said cellulose derivative is ethyl cellulose, methylcellulose, hydroxypropylmethylcellulose, hydroxypropyl cellulose, hydroxyethyl cellulose, hydroxymethyl cellulose, hydroxypropyl ethylcellulose, carboxy ethylcellulose, carboxymethyl ethylcellulose, carboxymethyl hydroxyethylcellulose, hydroxyethylmethyl carboxymethyl cellulose, hydroxyethyl methyl cellulose, carboxymethyl cellulose, methylhydroxyethyl cellulose, methylhydroxypropyl cellulose, carboxymethyl sulfoethyl cellulose, sodium carboxy methyl cellulose, cellulose acetate, cellulose acetate phthalate, cellulose acetate butyrate, hydroxypropylmethylcellulose acetate succinate, hydroxypropylmethylcellulose phthalate, hydroxymethyl ethylcellulose phthalate, cellulose acetate phthalate, cellulose acetate succinate, cellulose acetate maleate, cellulose acetate trimelliate, cellulose benzoate phthalate, cellulose propionate phthalate, methylcellulose phthalate, or ethylhydroxy ethylcellulose phthalate, or a combination thereof.

8. The formulation of claim 6 wherein said vinyl derivative or polymer or copolymer thereof is polyvinylacetate aqueous dispersion (Kollicoat® SR 30D), copolymers of vinyl pyrrolidone, polyvinyl alcohol phthalate, polyvinylacetal phthalate, polyvinyl butylate phthalate, polyvinylacetoacetal phthalate, or polyvinylpyrrolidone, or a combination thereof.

9. The formulation of claim 6 wherein said acrylic acid derivative is methacrylic acid, polymethacrylic acid, polyacrylate, or polymethacrylate, or a combination thereof, said polyacrylate being a) copolymer formed from monomers selected from the group consisting of methacrylic acid, methacrylic acid esters, acrylic acid and acrylic acid esters; b) copolymer formed from monomers selected from the group consisting of butyl methacrylate, (2-dimethylaminoethyl)methacrylate and methyl methacrylate; c) copolymer formed from monomers selected from the group consisting of ethyl acrylate, methyl methacrylate and trimethylammonioethyl methacrylate chloride; or d) copolymer of acrylate and methacrylates with/without quarternary ammonium group in combination with sodium carboxymethylcellulose.

10. The formulation of claim 5 wherein said non-polymeric pharmaceutically acceptable excipient is spermaceti wax, carnauba wax, Japan wax, bayberry wax, flax wax, beeswax, yellow wax, Chinese wax, shellac wax, lanolin wax, sugarcane wax, candelilla wax, castor wax, paraffin wax, microcrystalline wax, petrolatum wax, carbowax, mineral waxes, glyceryl monostearate, glyceryl distearate, glyceryl tristearate, glyceryl dipalmitate, glyceryl tripalmitate, glyceryl monopalmitate, glyceryl palmitostearate, glyceryl dilaurate, glyceryl trilaurate, glyceryl monolaurate, glyceryl didocosanoate, glyceryl tridocosanoate, glyceryl monodocosanoate, glyceryl monocaproate, glyceryl dicaproate, glyceryl tricaproate, glyceryl monomyristate, glyceryl dimyristate, glyceryl trimyristate, glyceryl monodecenoate, glyceryl didecenoate, glyceryl tridecenoate, glyceryl behenate, polyglyceryl diisostearate, lauroyl macrogolglycerides, oleoyl macrogolglycerides, stearoyl macrogolglycerides, hydrogenated palm kernel oil, hydrogenated peanut oil, hydrogenated palm oil, hydrogenated rapeseed oil, hydrogenated rice bran oil, hydrogenated soybean oil, hydrogenated sunflower oil, hydrogenated castor oil, hydrogenated cottonseed oil, decenoic acid, docosanoic acid, stearic acid, palmitic acid, lauric acid, myristic acid, cetyl alcohol, or stearyl alcohol, or a combination thereof.

11. (canceled)

12. The formulation of claim 5 wherein said carbomer is carbomer 934, carbomer 971, or carbomer 974, or a combination thereof.

13. The formulation of claim 5 wherein said ion exchange resin is a cationic ion exchange resin.

14. The formulation of claim 5 wherein said cyclodextrin is α-cyclodextrin, β-cyclodextrin, γ-cyclodextrin, dimethyl-β-cyclodextrin, trimethyl-β-cyclodextrin, hydroxymethyl-β-cyclodextrin, hydroxypropyl-β-cyclodextrin, hydroxyethyl-β-cyclodextrin, β-cyclodextrin sulfate, β-cyclodextrin sulfonate, or β-cyclodextrin sulfobutyl ether, dihydroxypropyl-β-cyclodextrin, glucosyl-α-cyclodextrin, glucosyl-β-cyclodextrin, diglucosyl-β-cyclodextrin, maltosyl-γ-cyclodextrin, maltosyl-γ-cyclodextrin, maltosyl-γ-cyclodextrin, maltotriosyl-βcyclodextrin, maltotriosyl-γ-cyclodextrin, dimaltosyl-β-cyclodextrin, maltosyl-β-cyclodextrin/dimaltosyl-β-cyclodextrin, or a combination thereof.

15. (canceled)

16. The formulation of claim 5 wherein said taste-masking agent is in the range of about 1% to about 95% by weight of the formulation.

17. The formulation of claim 5 wherein said taste-masking agent is in the range of about 2% to about 75% by weight of the formulation.

18. The formulation of claim 5 wherein said taste-masking agent is in the range of about 5% to about 60% by weight of the formulation.

19. The formulation of claim 1 wherein said influenza antiviral is taste-masked by physical mixing, coating, complexation, adsorption or salt formation using said taste-masking agent.

20. The formulation of claim 1 wherein said excipient is a binder, disintegrant, superdisintegrant, diluent, salivating agent, surfactant, flavor, sweetener, colorant, souring agent, viscolizer, glidant, lubricant, solubilizer, or stabilizer.

21. The formulation of claim 1 wherein said influenza antiviral is in the form of powder, granules, pellets, beads, minitablets, or a combination thereof

22. The formulation of claim 1 wherein said formulation is in the form of a dispersible tablet, orally disintegrating tablet, effervescent tablet, chewable tablet, sprinkle granules, dry suspension or dry syrup for reconstitution, quick melt wafers, lozenge, or chewing gum.

23. The formulation of claim 1 further comprising an additional active agent, said active agent being nelfinavir, abacavir, acemetacin, acetoniode, acetyl salicylic acid, aciclovir, acrivastine, acyclovir, adefovir, alclofenac, alminoprofen, amastatin, amprenavir, antipain, aprotinin, arbidol, astemizole, atazanavir, atorvastatin, atripla, azapropazone, azelastine, benorylate, benoxaprofen, betastatin, boceprevir, bucloxic acid, budesonide, caffeine, carprofen, celecoxib, cerivastatin, cetirizine, choline magnesium trisalicylate, cidofovir, clidanac, clopinac, combivir, darunavir, delavirdine, deracoxib, desloratadine, diclofenac, didanosine, diflunisal, docosanol, droxicam, ebastine, edoxudine, efavirenz, elastatinal, emtricitabine, enfuvirtide, entecavir, epinastine, etodolac, etoricoxib, famciclovir, fenbufen, fenclofenec, fenoprofen, fentiazac, feprazone, fexofenadine, floctafenine, flufenamic acid, flufenisal, fluprofen, flurbiprofen, fluticosone propionate, fluvastatin, fomivirsen, fosamprenavir, foscarnet, fosfonet, furofenac, ganciclovir, ibacitabine, ibufenac, ibuprofen, idoxuridine, imiquimod, imunovir, indinavir, indomethacin, indoprofen, isoxepac, isoxicam, ketoprofen, ketorolac, lamivudine, leupeptin, licofelone, lopinavir, lortadine, lovastatin, loviride, lumiracoxib, maraviroc, meclofenamic acid, mefenamic acid, miroprofen, mizolastine, mometasone furoate mononhydrate, moroxydine, nabumetone, naproxen, nelfinavir, nevirapine, nexavir, niflumic acid, norastemizole, oxaprozin, oxipinac, oxyphenbutazone, parecoxib, penciclovir, pepstatin, peramivir, phenylbutazone, piprofen, piroxicam, pirprofen, pleconaril, prapoprofen, pravastatin, proglumetacin, prometazine, raltegravir, ribavirin, ritonavir, rofecoxib, saquinavir, simvastatin, stavudine, sudoxicam, sulindac, suprofen, tenofovir, tenoxicam, terfenadine, tepoxalin, thiophosphonoformic acid, tiaprofenic acid, tiopinac, tioxaprofen, tipranavir, tolfenamic acid, tolmetin, triamcinalone, trifluridine, trizivir, tromantadine, truvada, valaciclovir, valdecoxib, valganciclovir, vicriviroc, vidarabine, viramidine, zalcitabine, zidometacin, zidovudine, zomepirac, ceftriaxone, cefotaxime, vancomycin, meropenem, cefepime, ceftazidime, cefuroxime, nalcillin, oxacillin, ampicillin, ticarcillin, ticarcillin/clavulinic acid, ampicillin/sulbactam, trimethoprim-sulfamethoxazole, clindamycin, synercid, amoxicillin, amoxicillin/clavulinic acid, cefuroxime, trimethoprim/ sulfamethoxazole, azithromycin, dicloxacillin, ciprofloxacin, levofloxacin, cefixime, cetpodoxime, loracarbef, cefadroxil, cefabutin, cefdinir or cephradine.

24. (canceled)
